(19) United States
(12) Patent Application Publication
(10) Pub. No.: US 2009/0220554 A1
(43) Pub. Date: Sep. 3, 2009

(54) Transglutaminase Inhibitors and Methods of Use Thereof

(76) Inventors: John Griffin, Atherton, CA (US); Guido Lanza, San Francisco, CA (US); Paul Boardman, South San Francisco, CA (US); Andrew Spencer, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/091,639

(22) PCT Filed: Oct. 25, 2006
(86) PCT No.: PCT/US06/41849

§ 371 Date: Sep. 9, 2008

Related U.S. Application Data
(60) Provisional application No. 60/730,302, filed on Oct. 25, 2005.

(51) Int. Cl. A61K 9/00; A61K 31/53; A61K 31/4704; A61K 31/497; A61K 31/404; A61K 31/4439; A61P 35/00; A61K 31/4709; A61K 31/513; A61K 38/48

(52) U.S. Cl. 424/400; 514/243; 514/312; 514/254.09; 514/418; 514/340; 514/414; 514/274; 424/94.63

(57) ABSTRACT

Transglutaminase inhibitors and methods of use thereof are provided.

### BACKGROUND OF THE INVENTION

Transglutaminases belong to a family of enzymes that play important roles in diverse biological functions by selectively cross-linking proteins. They catalyze formation of ε-(γ-glutamyl)-lysine cross-links between proteins, and may also incorporate polyamines into suitable protein substrates. This covalent isopeptide cross-link is stable and resistant to proteolysis, thereby increasing the resistance of tissue to chemical, enzymatic, and mechanical disruption. Among the members of this family are plasma transglutaminase, factor XIIIa, which stabilizes fibrin clots; keratinocyte transglutaminase and epidermal transglutaminase, which cross-link proteins on the outer surface of squamous epithelia; and tissue transglutaminase, which cross-links fibronectin in the extracellular matrix of organs such as brain, liver and the intestine.

Transglutaminase 2 (TG2, also known as tissue transglutaminase), a calcium-dependent member of the transglutaminase family, is reported to have extracellular as well as intracellular functions. Outside the cell, TG2 plays a crucial role in shaping the extracellular matrix by cross-linking fibronectin and related proteins. TG2 also promotes cell adhesion and motility by forming non-covalent complexes with other key proteins such as integrins and fibronectin. Intracellular TG2 loses enzyme activity when bound to GTP, but functions as a G-protein in the phospholipase C signal transduction cascade. Human TG2 is a structurally and mechanistically complex protein. Its catalytic mechanism is similar to that employed by cysteine proteases, involving a catalytic triad of cysteine, histidine, and aspartate. The cysteine thiol group reacts with a glutamine sidechain of a protein substrate to form a reactive thioester intermediate, from which the acyl group is transferred to another amine substrate.

Several members of the transglutaminase family have been linked to disease, including tissue transglutaminase (TG2), and the skin transglutaminases, TG1 and TG3. TG2 is a cytoplasmic enzyme present in many cells, including those in the blood vessel wall. Aberrant TG2 activity is believed to play a role in neurological disorders such as Alzheimer's, Parkinson's and Huntington's disease (see, for example, Kim et al. (2002) Neurochem. Int. 40:85-103; Karpuj et al. (2002) Nature Med. 8, 143-149). In Celiac Sprue, where TG2 is the predominant autoantigen, its pivotal role in unmasking antigenic epitopes by site specific deamidation of gluten peptides is well established. Expression of TG1 and TG2 have been correlated with various types of malignancies (see Zhang et al. (2003) Glia 42:194-208; and Martinet et al. (2003) Am. J. Respir. Cell. Mol. Biol. 28, 428-435), including glioblastomas, lung and breast cancers, suggesting an important role for TG2 in tumor proliferation and survival. Taken together, the above findings make a strong case for suitable small molecule TG2 inhibitors as experimental therapeutic agents. The medicinal attractiveness of this protein target is underscored by the observation that TG2 knockout mice are normal, lacking developmental, physiological or reproductive defects.

Although a number of TG2 inhibitors have been used in biological studies over the past two decades, many of these compounds (e.g. monodansyl cadaverine) contain primary amines in addition to potential inhibitory motifs, and it remains unclear whether the observed effects are due to excess competing amines or by blockage of TG2 substrate turnover. A few studies have utilized a suicide inhibitor, L682777, which inhibits human TG2 (Lorand et al. (1998) Exp Eye Res. 66:531-6). However, L682777 was designed as a specific inhibitor of Factor XIIIa, and is therefore unsuitable for evaluating TG2 biology in vivo. More recently, mechanism-based active-site inhibitors of guinea pig and human (Hausch et al. (2003) Chem Biol 10, 225-231; Choi et al. (2005) Chem. Biol. 12, 469-475) TG2 have been reported.

In view of the serious and widespread nature of Celiac Sprue and the difficulty of removing gluten from the diet, better methods of treatment are of great interest. In particular, there is a need for treatment methods that allow the Celiac Sprue individual to eat gluten-containing foodstuffs without ill effect or at least to tolerate such foodstuffs in small or moderate quantities without inducing relapse. The present invention meets this need for better therapies for Celiac Sprue by providing new drugs and methods and formulations of new and existing drugs to treat Celiac Sprue. International Patent Application US03/04743, herein specifically incorporated by reference, discloses aspects of gluten protease stability and immunogenicity.

TG2 has also been implicated in certain cancers. Neuro-oncological diseases including malignant neoplasms such as glioblastomas and melanomas metastatic to the brain are notoriously resistant to standard radiation and chemotherapy treatment. Current treatment strategies generally fail to achieve long-term survival. Similarly certain benign CNS tumors such as meningiomas are resistant to chemotherapy and radiation. Current treatment strategies with these tumors typically require major surgical resections or treatment with radiation in an attempt to control growth of recurrent or non-resectable tumors. Meningiomas are generally resistant to radiation-induced cell death and to chemotherapy. The mechanisms responsible for the failure of these brain tumors to respond to chemotherapy and radiation are not known. Therefore, identification of agents that augment sensitivity to chemotherapy and radiation therapy is important for improving treatment strategies in patients with these and other refractory cancers.

### SUMMARY OF THE INVENTION

The present invention provides methods that utilize administration of specific transglutaminase inhibitors provided herein to a patient for the treatment of conditions associated with undesirable transglutaminase activity. The inhibition of tissue transglutaminase (TG2) is of particular interest.

In one embodiment, the invention provides pharmaceutical formulations comprising a TG2 inhibitor of the invention and a pharmaceutically acceptable carrier. In certain embodiments, the formulation also comprises one or more glutenases, as described in U.S. Provisional Application 60/392,782 filed Jun. 28, 2002; and U.S. Provisional Application 60/428,033, filed Nov. 20, 2002, both of which are incorporated herein by reference. In other embodiments, the formulation comprises a chemotherapeutic agent.

The invention also provides methods for the administration of enteric formulations of one or more TG2 inhibitors of the invention to treat Celiac Sprue.

In another aspect, the TG2 inhibitors and/or pharmaceutical formulations of the present invention are useful in treating cancer, including neurologic cancers, such as gliomas, astrocytomas, meningiomas (which are cancers of neural crest-derived cells), etc., and other cancers, including melanoma, as well as other neurological disorders including Alzheimer's and Huntington's diseases, where the TGases appear to be a factor in the formation of inappropriate proteinaceous aggregates. The TG2 inhibitors act on some cancers to sensitize the tumor cells to killing by chemotherapeutic agents and/or radiation.

These and other aspects and embodiments of the invention and methods for making and using the invention are described in more detail in the description of the drawings and the invention, the examples, the claims, and the drawings that follow.

### DETAILED DESCRIPTION OF THE EMBODIMENTS

Transglutaminase inhibitors of the invention are administered to a patient for the treatment of conditions associated with undesirable transglutaminase activity, including Celiac Sprue, dermatitis herpetiformis, cancer, and neurological disorders including Alzheimer's and Huntington's diseases.

In some embodiments, particularly for the treatment of Celiac Sprue, the compositions include formulations of TG2 inhibitors of the invention that comprise an enteric coating that allows delivery of the agents to the intestine in an active form; the agents are stabilized to resist digestion or alternative chemical transformations in acidic stomach conditions. In another embodiment, food is pretreated or combined with glutenase, or a glutenase is co-administered (whether in time or in a formulation of the invention) with a TG2 inhibitor of the invention.

For the treatment of cancer, the TG2 inhibitors can act as a sensitizing agent, which enhances killing by a second agent, e.g. radiation, cytotoxic drugs, and the like. For sensitization, the TG2 inhibitor may be administered separately or in a co-formulation with a cytotoxic agent. Although the cytotoxic agents can be active when administered alone, the concentrations required for a therapeutic dose may create undesirable side effects. The combination therapy may provide for a therapeutic effect with less toxicity.

The subject methods are useful for both prophylactic and therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention. Such treatment is desirably performed prior to loss of function in the affected tissues; consequently, the prophylactic therapeutic benefits provided by the invention are also important. For example, treatment of a cancer patient may be reduction of tumor size, elimination of malignant cells, prevention of metastasis, or the prevention of relapse in a patient who has been put into remission.

### Disease Conditions

A number of pathological conditions have been associated with undesirable activity of tissue transglutaminases. For the most part, the disease-associated transglutaminases of interest for the present invention are TG1, TG2, and TG3. Conditions may involve over-expression of the transglutaminase, expression of transglutaminase in tissues or sites where it is not normally expressed, or may involve normal expression of transglutaminase in a disease context.

Diseases associated with transglutaminase include, inter alia, celiac sprue; dermatitis herpetiformis; inclusion body myositis; atherosclerosis; Alzheimer's disease; Huntington's disease; Parkinson's disease; progressive supranuclear palsy; and tumors, e.g. glioblastomas, meningioma, melanoma, etc.

Celiac Sprue is characterized by damage to the upper small intestine, causing effacement of the villi to produce a characteristically flat mucosa with markedly hypertrophic crypts. Clinical symptoms of Celiac Sprue include fatigue, chronic diarrhea, malabsorption of nutrients, weight loss, abdominal distension, anemia, as well as a substantially enhanced risk for the development of osteoporosis and intestinal malignancies (lymphoma and carcinoma). The disease has an incidence of approximately 1 in 200 in European populations. Therapeutic effect for Celiac Sprue is measured in terms of clinical outcome, or by immunological or biochemical tests. Suppression of the deleterious T-cell activity can be measured by enumeration of reactive Th1 cells, by quantitating the release of cytokines at the sites of lesions, or using other assays for the presence of autoimmune T cells known in the art. Also both the physician and patient can identify a reduction in symptoms of a disease. Evidence of therapeutic effect may be any diminution in the severity of disease, particularly diminution of the severity of such symptoms as fatigue, chronic diarrhea, malabsorption of nutrients, weight loss, abdominal distension, and anemia. Other disease indicia include the presence of antibodies specific for glutens, antibodies specific for tissue transglutaminase, the presence of pro-inflammatory T cells and cytokines, and degradation of the villus structure of the small intestine. Application of the methods and compositions of the invention can result in the improvement of any and all of these disease indicia of Celiac Sprue. Patients that can benefit from the present invention include both adults and children. Children in particular benefit from prophylactic treatment, as prevention of early exposure to toxic gluten peptides can prevent development of the disease into its more severe forms. Children suitable for prophylaxis in accordance with the methods of the invention can be identified by genetic testing for predisposition, e.g. by HLA typing; by family history, and by other methods known in the art. As is known in the art for other medications, and in accordance with the teachings herein, dosages of the TG2 inhibitors of the invention can be adjusted for pediatric use.

A related disease is dermatitis herpetiformis, which is a chronic eruption characterized by clusters of intensely pruritic vesicles, papules, and urticaria-like lesions. IgA deposits occur in almost all normal-appearing and perilesional skin. Asymptomatic gluten-sensitive enteropathy is found in 75 to 90% of patients and in some of their relatives. Onset is usually gradual. Itching and burning are severe, and scratching often obscures the primary lesions with eczematization of nearby skin, leading to an erroneous diagnosis of eczema.

Gluten proteins, which may include gliadins, secalins and hordeins, contain several sequences rich in Pro-Gln residues that are high-affinity substrates for TG2. TG2 is thought to be responsible for generating neoepitopes of gluten peptides through deamidation of glutamine residues. Presentation of these deamidated epitopes by DQ2 positive antigen presenting cells effectively stimulates proliferation of gliadin-specific T cells from intestinal biopsies of most Celiac Sprue patients. The toxic effects of gluten include immunogenicity of the gluten oligopeptides, leading to inflammation. TG2 also cross-links itself onto gliadin in vitro, and the cross-linked TGase might act as a hapten for the formation of antibodies against gluten peptides. In addition to its role as an extracellular enzyme, the protein is also thought to play a role in cell signaling across the plasma membrane by binding to integrins. By reacting with gluten peptides, a conformational change may be induced in TG2 that in turn leads to altered cell signaling and hence gluten mediated pathogenesis.

In addition to these conditions, it has been suggested that transglutaminase generates autoantibodies in a variety of autoimmune disorders by cross-linking potential autoantigens and acting as a hapten, e.g. in lupus, myasthenia gravis, multiple sclerosis, rheumatoid arthritis, etc. Anti-TG2 antibodies have been reported in lupus; and TG2 has been detected in the synovial fluid of arthritis patients, and the serum and cerebral spinal fluid of amyotrophic lateral sclerosis patients (see review by Kim et al. (2002) Neurochemistry International 40:85-103).

Inclusion body myositis is a progressive muscle disorder that affects older individuals. It is considered to be an autoimmune disease; associated with the expression of specific HLA molecules and a variety of autoantibodies. This disease is characterized by a progressively worsening weakness in the proximal and distal limbs that is resistant to steroid therapy. The histological features of this disease include distinctive rimmed vacuoles and filamentous inclusions, as well as mononuclear infiltrates, which consist predominantly of cytotoxic T cells. Total transglutaminase enzyme activity is elevated by 16-fold in diseased tissue, which is reflected in an increased number of ε(γ-glutamyl)lysine cross-links. This activity is apparently due to the increased expression of TG1 and TG2, which co-localize with deposits in vacuolated muscle fibers from patients with inclusion body myositis. The chronic inflammation that characterizes inclusion body myositis may also stimulate the expression of TG1 and TG2 and thereby contribute to the progressive nature of this disease.

In addition to autoimmune diseases, transglutaminases are associated with neurological conditions. Alzheimer's disease is associated with the selective loss of neurons in the neocortex, hippocampus, and amygdala, resulting in an impaired cognitive ability. This disease is also characterized by the presence of two types of protein aggregates: extracellular neuritic senile plaques, and intraneuronal neurofibrillary tangles. TGases are likely to contribute to the formation of these aggregates. Isoforms of TGase co-localize with the plaques and tangles in the brains of Alzheimer's disease patients and the number of ε(γ-glutamyl)lysine linkages in insoluble proteins from Alzheimer's disease brains is 30-50 times greater than the number found in normal brain tissues. In addition, total TGase activity is elevated in the affected areas of Alzheimer's disease brains, particularly TG1 and TG2. The components of plaques and tangles are substrates for TGases. For example, β-amyloid, which is found in a fibrillular form in plaques, is a substrate for TGases. Tau, the major component of the paired helical filaments that make up neurofibrillary tangles is also a substrate for TG2.

Huntington's disease is characterized by progressive motor and psychiatric disorders, as well as dementia. The most common manifestation of this disease is chorea: involuntary and non-directed motions that disrupt all normal activities eventually leading to death. The clinical progression of Huntington's disease is accompanied by specific neuronal loss and dysfunction, particularly in the striatum and later in the cerebral cortex. Huntington's disease is an autosomal dominant disease. The mutated gene and its product have been identified, and the protein found to be a substrate for TG2. Pathological mutations of the huntingtin gene, involving expansion of CAG repeats, result in stretches of polyglutamines of greater than 39 contiguous glutamine residues. The age of disease onset correlates inversely with the length of the polyglutamine expansion beyond the normal range, and there is a decrease in the age of disease onset with succeeding generations. There is elevated transglutaminase activity in the affected regions of diseased brains, including the striata. TG1, TG2 and TG3 are present in human brains, and all are elevated in Huntington's disease patient brains.

Parkinson's disease is characterized by tremor, bradykinesia, rigidity and postural instability. These motor disorders result primarily from a loss of dopaminergic neurons of the nigro-striatal pathway. Histologically, Parkinson's disease is characterized by a widespread distribution of Lewy bodies, which are intracytoplasmic aggregates of between 5 and 25 μm in diameter that feature a dense eosinophilic core and pale surrounding halo. Although Lewy bodies are thought to play a causative role in Parkinson's disease, these structures also have been identified in some cases of Alzheimer's disease. The major component of Lewy bodies is α-synuclein. A fragment of α-synuclein, known as the non-amyloid component (NAC), has been detected in the Lewy bodies of Parkinson's disease patients and the neuritic plaques of Alzheimer's disease patients. NAC is also as a substrate for transglutaminases, and is neurotoxic to primary dopaminergic neurons, as well as to neuroblastoma cells. TGases catalyze the formation of NAC polymers and aggregates of NAC and β-amyloid peptides.

Progressive supranuclear palsy is a motor disorder that initially presents in patients having unexpected falls. The later manifestations of the disease present as postural instability, vertical gaze palsy, axial rigidity, dysarthria and dementia. The most common pathological findings in this disease are midbrain atrophy with dilation of the aqueduct of Sylvius and depigmentation of the substantia nigra. These features are associated with the loss of neurons in the substantia nigra, globus pallidus, subthalamic nucleus, basal ganglia, diencephalon and brain stem. Neurofibrillary tangles occur in the affected areas of progressive supranuclear palsy patients. Tau, which is a substrate for transglutaminase, has also been identified as a major component of the detergent-insoluble proteins isolated from the brains of progressive supranuclear palsy patients. These aggregates also contain ε(γ-glutamyl)lysine cross links. Expression of TG1 and TG2, particularly TG1, are elevated in both the cerebellum of progressive supranuclear palsy patients.

Expression of transglutaminase has also been implicated in certain cancers, including glioblastoma, lung cancer, and cervical cancer. Hilton et al. ((1997) Neuropathol Appl Neurobiol. 23(6):507-11) found expression of tissue transglutaminase in fibrillary astrocytomas, anaplastic astrocytomas and glioblastomas. Labeling was particularly prominent in the pseudopalisading tumour cells that surrounded foci of necrosis and apoptosis in glioblastomas. Strong transglutaminase labeling was also observed in the endothelial cells of vessels showing microvascular proliferation in all of the glioblastomas studied. Enhanced expression of transglutaminase by endothelial cells in glioblastomas may contribute to the high prevalence of vascular thrombosis and necrosis in these tumours. In cervical cancers, TG1 is overexpressed (see, for example, Friedrich et al. (1999) Histochem J. 31(1):13-8).

Cancer, as used herein, refers to hyperproliferative conditions. The term denotes malignant as well as non-malignant cell populations. Such disorders have an excess cell proliferation of one or more subsets of cells, which often appear to differ from the surrounding tissue both morphologically and genotypically. The excess cell proliferation can be determined by reference to the general population and/or by reference to a particular patient, e.g. at an earlier point in the patient's life. Hyperproliferative cell disorders can occur in different types of animals and in humans, and produce different physical manifestations depending upon the affected cells.

Cancers include leukemias, lymphomas (Hodgkins and non-Hodgkins), sarcomas, melanomas, adenomas, carcinomas of solid tissue including breast cancer and pancreatic cancer, hypoxic tumors, squamous cell carcinomas of the mouth, throat, larynx, and lung, genitourinary cancers such as cervical and bladder cancer, hematopoietic cancers, head and neck cancers, and nervous system cancers, benign lesions such as papillomas, and the like. Cancers that form solid tumors, i.e. other than leukemias and lymphomas, are of interest.

Cancers of particular interest are neurologic cancers, including brain tumors. Neurologic tumors are classified according to the kind of cell from which the tumor seems to originate. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). WHO grade II astocytomas are the most indolent of the diffuse astrocytoma spectrum. Astrocytomas display a remarkable tendency to infiltrate the surrounding brain, confounding therapeutic attempts at local control. These invasive abilities are often apparent in low-grade as well as high-grade tumors.

Glioblastoma multiforme is the most malignant stage of astrocytoma, with survival times of less than 2 years for most patients. Histologically, these tumors are characterized by high proliferation indices, endothelial proliferation and focal necrosis. The highly proliferative nature of these lesions likely results from multiple mitogenic effects. One of the hallmarks of GBM is endothelial proliferation. A host of angiogenic growth factors and their receptors are found in GBMs.

There are biologic subsets of astrocytomas, which may reflect the clinical heterogeneity observed in these tumors. These subsets include brain stem gliomas, which are a form of pediatric diffuse, fibrillary astrocytoma that often follow a malignant course. Brain stem GBMs share genetic features with those adult GBMs that affect younger patients. Pleiomorphic xanthoastrocytoma (PXA) is a superficial, low-grade astrocytic tumor that predominantly affects young adults. While these tumors have a bizarre histological appearance, they are typically slow-growing tumors that may be amenable to surgical cure. Some PXAs, however, may recur as GBM. Pilocytic astrocytoma is the most common astrocytic tumor of childhood and differs clinically and histopathologically from the diffuse, fibrillary astrocytoma that affects adults. Pilocytic astrocytomas do not have the same genomic alterations as diffuse, fibrillary astrocytomas. Subependymal giant cell astrocytomas (SEGA) are periventricular, low-grade astrocytic tumors that are usually associated with tuberous sclerosis (TS), and are histologically identical to the so-called “candle-gutterings” that line the ventricles of TS patients. Similar to the other tumorous lesions in TS, these are slowly-growing and may be more akin to hamartomas than true neoplasms. Desmoplastic cerebral astrocytoma of infancy (DCAI) and desmoplastic infantile ganglioglioma (DIGG) are large, superficial, usually cystic, benign astrocytomas that affect children in the first year or two of life.

Oligodendrogliomas and oligoastrocytomas (mixed gliomas) are diffuse, primarily CNS glial tumors that are clinically and biologically most closely related to the diffuse, fibrillary astrocytomas. The tumors, however, are far less common than astrocytomas and have generally better prognoses than the diffuse astrocytomas. Oligodendrogliomas and oligoastrocytomas may progress, either to WHO grade III anaplastic oligodendroglioma or anaplastic oligoastrocytoma, or to WHO grade IV GBM. Thus, the genetic changes that lead to oligodendroglial tumors constitute yet another pathway to GBM.

Ependymomas are a clinically diverse group of gliomas that vary from aggressive intraventricular tumors of children to benign spinal cord tumors in adults. Transitions of ependymoma to GBM are rare. Choroid plexus tumors are also a varied group of tumors that preferentially occur in the ventricular system, ranging from aggressive supratentorial intraventricular tumors of children to benign cerebellopontine angle tumors of adults. Choroid plexus tumors have been reported occasionally in patients with Li-Fraumeni syndrome and von Hippel-Lindau (VHL) disease.

Medulloblastomas are malignant, primitive tumors that arise in the posterior fossa, primarily in children. These tumors also occur in young adults. Medulloblastomas often are surgically resected with subsequent treatment with chemotherapy and/or radiation. They may recur locally or occasionally as drop metastasis from the posterior fossa to the spine. Meningiomas are common intracranial tumors that arise in the meninges and compress the underlying brain. Although typically considered benign and only rarely frankly malignant, management of these tumors often poses clinical challenges. Histological grades of meningiomas vary with the majority benign, WHO grade I/IV (82%); less commonly atypical, WHO II/IV (15%); and infrequently they occur as anaplastic or malignant, WHO grade III/IV (3%).

Schwannomas are benign tumors that arise on peripheral nerves. Schwannomas may arise on cranial nerves, particularly the vestibular portion of the eighth cranial nerve (vestibular schwannomas, acoustic neuromas) where they present as cerebellopontine angle masses. Hemangioblastomas are tumors of uncertain origin that are composed of endothelial cells, pericytes and so-called stromal cells. These benign tumors most frequently occur in the cerebellum and spinal cord of young adults. Multiple hemangioblastomas are characteristic of von Hippel-Lindau disease (VHL). Hemangiopericytomas (HPCs) are dural tumors which may display locally aggressive behavior and may metastasize. The histogenesis of dural-based hemangiopericytoma (HPC) has long been debated, with some authors classifying it as a distinct entity and others classifying it as a subtype of meningioma.

The symptoms of both primary and metastatic brain tumors often depend on the location in the brain and the size of the tumor. Since various regions of the brain are responsible for specific functions, clinical symptoms will vary a great deal. Tumors in the frontal lobe of the brain may cause weakness and paralysis, mood disturbances, difficulty thinking, confusion and disorientation, and wide emotional mood swings. Parietal lobe tumors may cause seizures, numbness or paralysis, difficulty with handwriting, inability to perform simple mathematical problems, difficulty with certain movements, and loss of the sense of touch. Tumors in the occipital lobe can cause loss of vision in half of each visual field, visual hallucinations, and seizures. Temporal lobe tumors can cause seizures, perceptual and spatial disturbances, and receptive aphasia. If a tumor occurs in the cerebellum, the person may have ataxia, loss of coordination, headaches, and vomiting. Tumors in the hypothalamus may cause emotional changes, and changes in the perception of hot and cold. In addition, hypothalamic tumors may affect growth and nutrition in children. With the exception of the cerebellum, a tumor on one side of the brain causes symptoms and impairment on the opposite side of the body.

The compounds described herein are useful in the treatment of individuals suffering from the conditions described above, by administering an effective dose of a TG2 inhibitor, through a pharmaceutical formulation, and the like. Diagnosis of suitable patients may utilize a variety of criteria known to those of skill in the art.

Compounds of interest for inhibition of TG2 include compounds of Formulas I-III and V-IX:

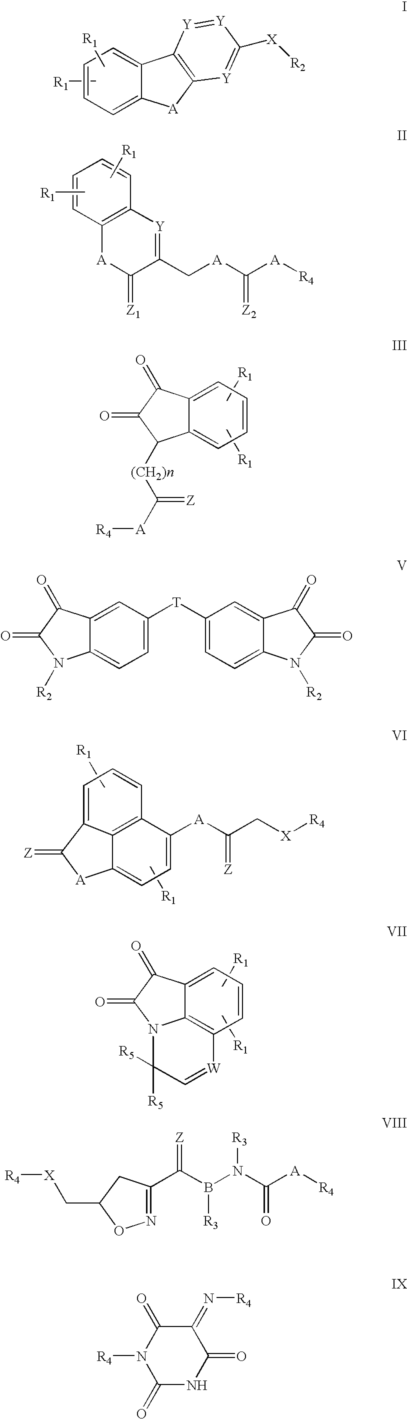

In Formula I, R₁ is in each instance independently selected from hydrogen, halogen, nitro, sulfonyl, acyl, alkoxy, alkyl, etc. R₂ is alkyl, aryl, heteroaryl or preferably an acetic ester (CH₂C(O)OR₃) or an acetamide (CH₂C(O)NR₃R₃). R₃ is H, optionally substituted lower alkyl, aryl, heteroaryl, alkaryl. A is O, S or preferably N—R₃. Y is in each instance independently C—R₁ or N. X is CH₂, CF₂, CH(OH), CH(O-lower alkyl), C(O), N—R₃, O, S, S(O) or S(O)₂.

In some embodiments of Formula I, one R₁ group is H, the other R₁ group is attached para to the A group, and is preferably halo or lower alkyl, including methyl, ethyl, etc.; A is NH; X is S; at least one Y is N, and in some embodiments each Y is N; and R₂ is an acetic ester (CH₂C(O)OR₃) or an acetamide (CH₂C(O)NR₃R₃.

In some embodiments of the invention, the compound of Formula I is CSRF-2 (6-fluoro-9H-1,3,4,9-tetraaza-fluoren-2-ylsulfanyl)-acetic acid cyclohexyl ester), e.g. in methods of treatment as set forth herein, and in pharmaceutical formulations. In other embodiments of the invention, the compound of Formula I is other than CSRF-2.

In Formula II, R₁ is in each instance independently selected from hydrogen, halogen, nitro, sulfonyl, acyl, alkoxy, alkyl, etc. R₃ is H, optionally substituted lower alkyl, aryl, heteroaryl, alkaryl. A is O, S or preferably N—R₃. Y is C—R₁ or N. Z₁ and Z₂ are in each instance independently O or S. R₄ is hydrogen, optionally substituted aryl, heteroaryl alkyl, alkaryl, etc.

In preferred embodiments of Formula II, each A is NH or N—R₃; Z₁ is O; Z₂ is S and R₄ is substituted aryl or heteroaryl.

In some embodiments of the invention, the compound of Formula II is CSRF-3 (1-(7,8-dimethyl-2-oxo-1,2-dihydro-quinolin-3-ylmethyl)-1-(tetrahydrofuran-2-ylmethyl)-3-m-tolyl-thiourea), e.g. in methods of treatment as set forth herein, and in pharmaceutical formulations. In other embodiments of the invention, the compound of Formula II is other than CSRF-3.

In Formula III, R₁ is in each instance independently selected from hydrogen, halogen, nitro, sulfonyl, acyl, alkoxy, alkyl, etc. R₃ is H, optionally substituted lower alkyl, aryl, heteroaryl, alkaryl. A is O, S or preferably N—R₃. Z is O or S. R₄ is hydrogen, optionally substituted aryl, heteroaryl alkyl, alkaryl, etc. n is 0 or 1.

In some embodiments of Formula III, n is O; Z is O; A is NH and R₄ is optionally substituted alkyl, cycloalkyl or aryl. In some embodiments, at least one R₁ is H, where the other R₁ is a substituent, e.g. halo, alkyl, etc. at position 5. In other embodiments of Formula III, n is 1; A is NH; Z is O; and R₄ is optionally substituted alkyl, cycloalkyl or aryl.

In some embodiments of the invention, the compound of Formula III is CSRF-6 (2-(2,3-dioxo-2,3-dihydro-indol-1-yl)-N-{4-[4-(thiophene-2-carbonyl)-piperazin-1-yl]-phenyl}-acetamide), CSRF-7 (2-(5-bromo-2,3-dioxo-2,3-dihydro-indol-1-yl)-N-cyclopropylmethyl-acetamide); CSRF-8 (2-(5-methyl-2,3-dioxo-2,3-dihydro-indol-1-yl)-N-{4-[tetrahydrofuran-2-ylmethyl)-sulfamoyl]-phenyl}-acetamide); or CSRF-20 (5-bromo-2,3-dioxo-2,3-dihydro-indole-1-carboxylic acid cyclohexylamide), e.g. in methods of treatment as set forth herein, and in pharmaceutical formulations. In other embodiments of the invention, the compound of Formula III is other than CSRF-6, CSRF-7, CSRF-8, or CSRF-20.

In Formula V, R₂ is alkyl, aryl, heteroaryl or preferably an acetic ester (CH₂C(O)OR₃) or an acetamide (CH₂C(O)NR₃R₃). T is CH₂, CF₂, CH(OH), CH(O-lower alkyl), C(O), N—R₃, O, S, S(O), S(O)₂ or a linker represented by the following formula: —X′-Z-(Y′-Z)ₘ-Y″-Z-X′- in which: m is an integer of from 0 to 20; X′ at each separate occurrence is —O—, —S—, —S(O)—, —S(O)₂—, —NR—, —N⁺RR′-, —C(O)—, —C(O)O—, —C(O)NH—, —C(S), —C(S)O—, —C(S)NH— or a covalent bond, where R and R′ at each separate occurrence are as defined below for R′ and R″; Z is at each separate occurrence selected from alkylene, substituted alkylene, alkylalkoxy, cycloalkylene, substituted cycloalkylene, alkenylene, substituted alkenylene, alkynylene, substituted alkynylene, cycloalkenylene, substituted alkenylene, arylene, substituted arylene, heteroarylene, heterocyclene, substituted heterocyclene, crown compounds, or a covalent bond; Y′ and Y′ at each separate occurrence are selected from —S—S— or a covalent bond;

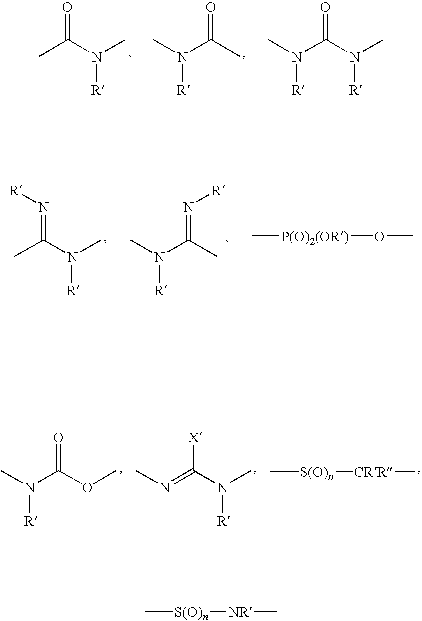

in which: n is 0, 1 or 2; and R′ and R″ at each separate occurrence are selected from hydrogen, alkyl, substituted alkyl, cycloalkyl, substituted cycloalkyl, alkenyl, substituted alkenyl, alkynyl, substituted alkynyl, aryl, heteroaryl or heterocyclic. For further descriptions of linkers, see U.S. Pat. Nos. 6,288,055 and 6,897,305, each of which is herein specifically incorporated by reference with respect to teaching of linkers and methods of synthesis thereof.

In some preferred embodiments of Formula V, L is a diradical comprising 2-12 heavy atoms selected from C, N, O, S, P and halogen. In further preferred embodiments, L is optionally substituted alkylene. In other further preferred embodiments, L comprises one or more amide, ester, ether, sulfonyl, sulfonamide or phosphoryl groups.

In some embodiments of the invention, the compound of Formula V is CSRF-14 (1,1-di-(1H-indole-2,3-dione-5-yl)-methane) or CSRF-18 (di-(1H-indole-2,3-dione-5-yl)-ether), e.g. in methods of treatment as set forth herein, and in pharmaceutical formulations. In other embodiments of the invention, the compound of Formula V is other than CSRF-14 or CSRF-18.

In Formula VI, R₁ is in each instance independently selected from hydrogen, halogen, nitro, sulfonyl, acyl, alkoxy, alkyl, etc. R₃ is H, optionally substituted lower alkyl, aryl, heteroaryl, alkaryl. A is independently and in each instance O, S or preferably N—R₃. Z is independently and in each instance O or S. R₄ is hydrogen, optionally substituted aryl, heteroaryl alkyl, alkaryl, etc. X is CH₂, CF₂, CH(OH), CH(O-lower alkyl), C(O), N—R₃, O, S, S(O) or S(O)₂.

In some embodiments of Formula VI, one or both R₁ are hydrogen; A is NH or NR₃; Z is O and R₄ is optionally substituted aryl or heteroaryl.

In some embodiments of the invention, the compound of Formula VI is CSRF-17 (N-(1-ethyl-2-oxo-1,2-dihydro-benzo[cd]indol-6-yl)-quinolin-2-ylsulfanyl)-acetamide), e.g. in methods of treatment as set forth herein, and in pharmaceutical formulations. In other embodiments of the invention, the compound of Formula VI is other than CSRF-17.

In Formula VII, R₁ is in each instance independently hydrogen, halogen, nitro, sulfonyl, acyl, alkoxy, alkyl, etc. W is N or preferably C—R₃, where R₃ is H, optionally substituted lower alkyl, aryl, heteroaryl or alkaryl. R₅ is in each instance independently H, optionally substituted lower alkyl, aryl, heteroaryl, alkaryl, a carboxylic ester (C(O)OR₃) or a carboxamide (C(O)NR₃R₃).

In some embodiments of the invention, the compound of Formula VII is CSRF-24 (tetrahydrofuran-2-carboxylic acid 4,4,6-trimethyl-1,2-dioxo-1,2-dihydro-4H-pyrrolo[3,2,1-ij]quinolin-8-yl ester), e.g. in methods of treatment as set forth herein, and in pharmaceutical formulations. In other embodiments of the invention, the compound of Formula IX is other than CSRF-24.

In Formula VIII, R₁ is in each instance independently selected from hydrogen, halogen, nitro, sulfonyl, acyl, alkoxy, alkyl, etc. R₃ is H, optionally substituted lower alkyl, aryl, heteroaryl, alkaryl. A is O, S or preferably N—R₃. B is CH or N. Z is O or S. R₄ is independently and in each instance hydrogen, optionally substituted aryl, heteroaryl alkyl, alkaryl, etc. X is CH₂, CF₂, CH(OH), CH(O-lower alkyl), C(O), N—R₃, O, S, S(O) or S(O)₂.

In preferred embodiments of Formula VII, each A is N—R₃, X is NH, Z is O and R₄ is in each instance independently optionally substituted aryl or heteroaryl.

In some embodiments of the invention, the compound of Formula VIII is CSRF-13 (1-(4-chloro-phenyl)-3-(2-{5-[3-chloro-5-trifluoromethyl-pyridin-2-ylamino)-methyl]-4,5-dihydro-isoxazol-3-yl}-carboxamido-N-urea), e.g. in methods of treatment as set forth herein, and in pharmaceutical formulations. In other embodiments of the invention, the compound of Formula VIII is other than CSRF-13.

In Formula IX, R₄ is independently and in each instance hydrogen, optionally substituted aryl, heteroaryl alkyl, alkaryl, etc.

In preferred embodiments of Formula IX, R₄ is in each instance independently optionally substituted aryl or heteroaryl.

In some embodiments of the invention, the compound of Formula IX is CSRF-21 5-[4-Chloro-3-(1,3-dioxo-1,3-dihydro-isoindol-2-ylmethyl)-phenylimino]-1-(3-chloro-phenyl)-pyrimidine-2,4,6-trione, e.g. in methods of treatment as set forth herein, and in pharmaceutical formulations. In other embodiments of the invention, the compound of Formula VIII is other than CSRF-21.

The illustrative compounds of the invention described above were tested in a TG2 assay with recombinant human tissue transglutaminase, as described in the Examples. Competitive inhibition with respect to the Cbz-Gln-Gly substrate was observed for all substrates. It should be noted that, although competitive inhibition is observed upon addition of these compounds, this observation may result from the ability of some inhibitors to bind to the GTP binding site of TG2. By binding to the GTP binding site, the protein is maintained in a catalytically inactive state, and is thus unable to react with gluten peptides. Thus, it follows that pharmacologically useful inhibitors of TG2 could be identified based on their ability to bind to either the enzyme active site or the GTP binding site.

The TG2 inhibitors, or their pharmaceutically acceptable salts may contain one or more asymmetric centers and may thus give rise to enantiomers, diastereomers, and other stereoisomeric forms that may be defined, in terms of absolute stereochemistry, as (R) or (S) or, as (D) or (L) for amino acids. The present invention is meant to include all such possible isomers, as well as, their racemic and optically pure forms. Optically active (+) and (−), (R) and (S), or (D) and (L) isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

To facilitate an appreciation of the invention, the TG2 inhibitors of the invention have in part been described above with structures containing variable “R” groups that are defined by reference to the various organic moieties that can be present at the indicated position in the structure. Below, brief definitions are provided for the phrases used to define the organic moieties listed for each R group.

As used herein, “alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like. Unless stated otherwise specifically in the specification, the alkyl radical may be optionally substituted by hydroxy, alkoxy, aryloxy, haloalkoxy, cyano, nitro, mercapto, alkylthio, —N(R⁸)₂, —C(O)OR⁸, —C(O)N(R⁸)₂ or —N(R⁸)C(O)R⁸ where each R⁸ is independently hydrogen, alkyl, alkenyl, cycloalkyl, cycloalkylalkyl, aralkyl or aryl. Unless stated otherwise specifically in the specification, it is understood that for radicals, as defined below, that contain a substituted alkyl group that the substitution can occur on any carbon of the alkyl group.

“Alkoxy” refers to a radical of the formula —ORₐ where Rₐ is an alkyl radical as defined above, e.g., methoxy, ethoxy, n-propoxy, 1-methylethoxy (iso-propoxy), n-butoxy, n-pentoxy, 1,1-dimethylethoxy (t-butoxy), and the like. Unless stated otherwise specifically in the specification, it is understood that for radicals, as defined below, that contain a substituted alkoxy group that the substitution can occur on any carbon of the alkoxy group. The alkyl radical in the alkoxy radical may be optionally substituted as described above.

“Alkylthio” refers to a radical of the formula —SRₐ where Rₐ is an alkyl radical as defined above, e.g., methylthio, ethylthio, n-propylthio, 1-methylethylthio (iso-propylthio), n-butylthio, n-pentylthio, 1,1-dimethylethylthio (t-butylthio), and the like. Unless stated otherwise specifically in the specification, it is understood that for radicals, as defined below, that contain a substituted alkylthio group that the substitution can occur on any carbon of the alkylthio group. The alkyl radical in the alkylthio radical may be optionally substituted as described above.

“Alkenyl” refers to a straight or branched hydrocarbon chain radical consisting solely of carbon and hydrogen atoms, containing at least one double bond, having from two to eight carbon atoms, and which is attached to the rest of the molecule by a single bond or a double bond, e.g., ethenyl, prop-1-enyl, but-1-enyl, pent-1-enyl, penta-1,4-dienyl, and the like. Unless stated otherwise specifically in the specification, the alkenyl radical may be optionally substituted by hydroxy, alkoxy, haloalkoxy, cyano, nitro, mercapto, alkylthio, cycloalkyl, —N(R⁸)₂, —C(O)OR⁸, —C(O)N(R⁸)₂ or —N(R⁸)—C(O)—R⁸ where each R⁸ is independently hydrogen, alkyl, alkenyl, cycloalkyl, cycloalkylalkyl, aralkyl or aryl. Unless stated otherwise specifically in the specification, it is understood that for radicals, as defined below, that contain a substituted alkenyl group that the substitution can occur on any carbon of the alkenyl group.

“Aryl” refers to a phenyl or naphthyl radical. Unless stated otherwise specifically in the specification, the term “aryl” or the prefix “ar-” (such as in “aralkyl”) is meant to include aryl radicals optionally substituted by one or more substituents selected from the group consisting of hydroxy, alkoxy, aryloxy, haloalkoxy, cyano, nitro, mercapto, alkylthio, cycloalkyl, —N(R⁸)₂, —C(O)OR⁸, —C(O)N(R⁸)₂ or —N(R⁸)C(O)R⁸ where each R⁸ is independently hydrogen, alkyl, alkenyl, cycloalkyl, cycloalkylalkyl, aralkyl or aryl. The term “aryl” also refers to the compound C₆H₅, i.e. Bn.

“Aralkyl” refers to a radical of the formula —RₐR_{b} where Rₐ is an alkyl radical as defined above and R_{b} is one or more aryl radicals as defined above, e.g., benzyl, diphenylmethyl and the like. The aryl radical(s) may be optionally substituted as described above.

“Aralkenyl” refers to a radical of the formula —R_{c}R_{b} where R_{c} is an alkenyl radical as defined above and R_{b} is one or more aryl radicals as defined above, e.g., 3-phenylprop-1-enyl, and the like. The aryl radical(s) and the alkenyl radical may be optionally substituted as described above.

“Alkylene chain” refers to a straight or branched divalent hydrocarbon chain consisting solely of carbon and hydrogen, containing no unsaturation and having from one to eight carbon atoms, e.g., methylene, ethylene, propylene, n-butylene, and the like. The alkylene chain may be optionally substituted by one or more substituents selected from the group consisting of aryl, halo, hydroxy, alkoxy, haloalkoxy, cyano, nitro, mercapto, alkylthio, cycloalkyl, —N(R⁸)₂, —C(O)OR⁸, —C(O)N(R⁸)₂ or —N(R⁸)C(O)R⁸ where each R⁸ is independently hydrogen, alkyl, alkenyl, cycloalkyl, cycloalkylalkyl, aralkyl or aryl. The alkylene chain may be attached to the rest of the molecule through any two carbons within the chain.

“Alkenylene chain” refers to a straight or branched divalent hydrocarbon chain consisting solely of carbon and hydrogen, containing at least one double bond and having from two to eight carbon atoms, e.g., ethenylene, prop-1-enylene, but-1-enylene, pent-1-enylene, hexa-1,4-dienylene, and the like. The alkenylene chain may be optionally substituted by one or more substituents selected from the group consisting of aryl, halo, hydroxy, alkoxy, haloalkoxy, cyano, nitro, mercapto, alkylthio, cycloalkyl, —N(R⁸)₂, —C(O)OR⁸, —C(O)N(R⁸)₂ or —N(R⁸)C(O)R⁸ where each R⁸ is independently hydrogen, alkyl, alkenyl, cycloalkyl, cycloalkylalkyl, aralkyl or aryl. The alkenylene chain may be attached to the rest of the molecule through any two carbons within the chain.

“Cycloalkyl” refers to a stable monovalent monocyclic or bicyclic hydrocarbon radical consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group consisting of alkyl, aryl, aralkyl, halo, haloalkyl, hydroxy, alkoxy, haloalkoxy, cyano, nitro, mercapto, alkylthio, cycloalkyl, —N(R⁸)₂, —C(O)OR⁸, —C(O)N(R⁸)₂ or —N(R⁸)C(O)R⁸ where each R⁸ is independently hydrogen, alkyl, alkenyl, cycloalkyl, cycloalkylalkyl, aralkyl or aryl.

“Cycloalkylalkyl” refers to a radical of the formula —RₐR_{d} where Rₐ is an alkyl radical as defined above and R_{d} is a cycloalkyl radical as defined above. The alkyl radical and the cycloalkyl radical may be optionally substituted as defined above.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like.

“Haloalkoxy” refers to a radical of the formula —OR_{c}, where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

“Heterocyclyl” or “heteroaryl” refer to a stable 3- to 15-membered ring radical which consists of carbon atoms and from one to five heteroatoms selected from the group consisting of nitrogen, oxygen and sulfur. For purposes of this invention, the heterocyclyl or heteroaryl radical may be a monocyclic, bicyclic or tricyclic ring system, which may include fused or bridged ring systems; and the nitrogen, carbon or sulfur atoms in the heterocyclyl or heteroaryl radical may be optionally oxidized; the nitrogen atom may be optionally quaternized; and the heterocyclyl or heteroaryl radical may be aromatic or partially or fully saturated. The heterocyclyl or heteroaryl radical may not be attached to the rest of the molecule at any heteroatom atom. Examples of such heterocyclyl or heteroaryl radicals include, but are not limited to, azepinyl, acridinyl, benzimidazolyl, benzthiazolyl, benzothiadiazolyl, benzoxazolyl, benzodioxolyl, benzodioxinyl, benzopyranyl, benzopyranonyl, benzofuranyl, benzofuranonyl, benzothienyl (benzothiophenyl), benzotriazolyl, carbazolyl, cinnolinyl, decahydroisoquinolyl, dioxolanyl, furanyl, furanonyl, isothiazolyl, imidazolyl, imidazolinyl, imidazolidinyl, isothiazolidinyl, indolyl, indazolyl, isoindolyl, indolinyl, isoindolinyl, indolizinyl, isoxazolyl, isoxazolidinyl, morpholinyl, naphthyridinyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, oxiranyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydrofuryl, triazinyl, tetrahydropyranyl, thienyl, thiamorpholinyl, thiamorpholinyl sulfoxide, and thiamorpholinyl sulfone. Exemplary heteroaryl groups include benzofuran, benzothiophene, furan, imidazole, indole, isothiazole, oxazole, piperazine, pyrazine, pyrazole, pyridazine, pyridine, pyrimidine, pyrrole, quinoline, thiadiazole, thiazole and thiophene. Unless stated otherwise specifically in the specification, the term “heterocyclyl” or heteroaryl is meant to include heterocyclyl or heteroaryl radicals as defined above which are optionally substituted by one or more substituents selected from the group consisting of alkyl, halo, nitro, cyano, haloalkyl, haloalkoxy, aryl, heterocyclyl, heterocyclylalkyl, —OR⁸, —R⁷—OR⁸, —C(O)OR⁸, —R⁷—C(O)OR⁸, —C(O)N(R⁸)₂, —N(R⁸)₂, —R⁷—N(R⁸)₂, and —N(R⁸)C(O)R⁸ wherein each R⁷ is a straight or branched alkylene or alkenylene chain and each R⁸ is independently hydrogen, alkyl, alkenyl, cycloalkyl, cycloalkylalkyl, aralkyl or aryl.

“Heterocyclylalkyl” refers to a radical of the formula —RₐRₑ where Rₐ is an alkyl radical as defined above and Rₑ is a heterocyclyl or heteroaryl radical as defined above, and if the heterocyclyl or heteroaryl is a nitrogen-containing heterocyclyl or heteroaryl, the heterocyclyl or heteroaryl may be attached to the alkyl radical at the nitrogen atom. The heterocyclyl or heteroaryl radical may be optionally substituted as defined above.

In the formulas provided herein, molecular variations are included, which may be based on isosteric replacement. “Isosteric replacement” refers to the concept of modifying chemicals through the replacement of single atoms or entire functional groups with alternatives that have similar size, shape and electronic properties, e.g. O is the isosteric replacement of S, COOH is the isosteric replacement of tetrazole, F is the isosteric replacement of H, sulfonate is the isosteric replacement of phosphate etc.

As used herein, compounds which are “commercially available” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Il.), Riedel de Haen A G (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.), Novabiochem and Argonaut Technology.

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts. “Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (+) and (−), (R)- and (S)—, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The present invention provides the TG2 inhibitors in a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the TG2 inhibitors is achieved in various ways, although oral and intravenous administration are preferred routes. In some formulations, the TG2 inhibitors are systemic after administration; in others, the inhibitor is localized by virtue of the formulation, such as the use of an implant that acts to retain the active dose at the site of implantation.

In some pharmaceutical dosage forms, the TG2 inhibitors are administered in the form of their pharmaceutically acceptable salts. In some dosage forms, the TG2 inhibitor is used alone, while in others, the TG2 is used in combination with one or more additional pharmaceutically active compounds.

In combination therapies for the treatment of Celiac Sprue and/or Dermatitis Herpetiforms, the other active compound is, in some embodiments, a glutenase that can cleave or otherwise degrade a toxic gluten oligopeptide, as described in the Examples below.

In combination therapies for the treatment of cancer, the TG2 inhibitor may be combined with a cytotoxic agent, or administered in combination with radiation therapy. Cytotoxic agents that act to reduce cellular proliferation are known in the art and widely used. Such agents include alkylating agents, such as nitrogen mustards, e.g. mechlorethamine, cyclophosphamide, melphalan (L sarcolysin), etc.; and nitrosoureas, e.g. carmustine (BCNU), lomustine (CCNU), semustine (methyl-CCNU), streptozocin, chlorozotocin, etc.

Antimetabolite agents include pyrimidines, e.g. cytarabine (CYTOSAR-U), cytosine arabinoside, fluorouracil (5-FU), floxuridine (FUdR), etc.; purines, e.g. thioguanine (6-thioguanine), mercaptopurine (6-MP), pentostatin, fluorouracil (5-FU) etc.; and folic acid analogs, e.g. methotrexate, 10-propargyl-5,8-dideazafolate (PDDF, CB3717), 5,8-dideazatetrahydrofolic acid (DDATHF), leucovorin, etc.

Other natural products include azathioprine; brequinar; alkaloids and synthetic or semi-synthetic derivatives thereof, e.g. vincristine, vinblastine, vinorelbine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithromycin); anthracenediones, e.g. mitoxantrone; azirinopyrrolo indolediones, e.g. mitomycin; and the like.

Other chemotherapeutic agents include metal complexes, e.g. cisplatin (cis-DDP), carboplatin, etc.; ureas, e.g. hydroxyurea; and hydrazines, e.g. N-methylhydrazine. Other anti-proliferative agents of interest include immunosuppressants, e.g. mycophenolic acid, thalidomide, desoxyspergualin, azasporine, leflunomide, mizoribine, azaspirane (SKF 105685), etc.

The antineoplastic agents taxols (or taxanes) hyperstabilize polymerized microtubules, leading to mitotic arrest and cytotoxicity in proliferating cells. Taxanes (or taxols), such as paclitaxel, docetaxel, etc. are of interest. Also of interest are the microtubule stabilizing epothilones (see Bollag et al. (1995) Cancer Research, Vol 55, Issue 11 2325-2333, herein incorporated by reference with respect to teachings of the class, and use thereof of these chemotherapeutic agents), e.g. epothilone A and epothilone B.

Retinoids, e.g. vitamin A, 13-cis-retinoic acid, trans-retinoic acid, isotretinoin, etc.; carotenoids, e.g. beta-carotene, vitamin D, etc. Retinoids regulate epithelial cell differentiation and proliferation, and are used in both treatment and prophylaxis of epithelial hyperproliferative disorders.

Topoisomerase inhibitors of interest include irinotecan (CPT-11), a topoisomerase I inhibitor. Other topoisomerase inhibitors of interest in the subject methods include doxorubicin and carboplatinum, which inhibit type II topoisomerase.

In such embodiments for the treatment of cancer, a cytotoxic agent may be an alkylating agent such as BCNU or temozolomide, an antimitotic agent such as a taxane or epothilone, or arginine deiminase.

Pharmaceutical Formulations: The TG2 inhibitors can be incorporated into a variety of formulations for therapeutic administration. In combination therapies, the TG2 inhibitor and second agent can be delivered simultaneously, or within a short period of time, by the same or by different routes. In one embodiment of the invention, a co-formulation is used, where the two components are combined in a single suspension. Alternatively, the two may be separately formulated.

Part of the total dose may be administered by different routes. Such administration may use any route that results in systemic absorption, by any one of several known routes, including but not limited to inhalation, i.e. pulmonary aerosol administration; intranasal; sublingually; orally; and by injection, e.g. subcutaneously, intramuscularly, etc.

For injectables, the agents are used in formulations containing cyclodextrin, cremophor, DMSO, ethanol, propylene glycol, solutol, Tween, triglyceride and/or PEG. For oral preparations, the agents are used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and in some embodiments, with diluents, buffering agents, moistening agents, preservatives and flavoring agents.

In one embodiment of the invention, the oral formulations comprise enteric coatings, so that the active agent is delivered to the intestinal tract. Enteric formulations are often used to protect an active ingredient from the strongly acid contents of the stomach. Such formulations are created by coating a solid dosage form with a film of a polymer that is insoluble in acid environments and soluble in basic environments. Exemplary films are cellulose acetate phthalate, polyvinyl acetate phthalate, hydroxypropyl methylcellulose phthalate and hydroxypropyl methylcellulose acetate succinate, methacrylate copolymers, and cellulose acetate phthalate.

Other enteric formulations of the TG2 inhibitors of the invention comprise engineered polymer microspheres made of biologically erodable polymers, which display strong adhesive interactions with gastrointestinal mucus and cellular linings, can traverse both the mucosal absorptive epithelium and the follicle-associated epithelium covering the lymphoid tissue of Peyer's patches. The polymers maintain contact with intestinal epithelium for extended periods of time and actually penetrate it, through and between cells. See, for example, Mathiowitz et al. (1997) Nature 386 (6623): 410-414. Drug delivery systems can also utilize a core of superporous hydrogels (SPH) and SPH composite (SPHC), as described by Dorkoosh et al. (2001) J Control Release 71 (3):307-18.

In another embodiment, the TG2 inhibitor or formulation thereof is admixed with food, or used to pre-treat foodstuffs containing glutens.

Formulations are typically provided in a unit dosage form, where the term “unit dosage form,” refers to physically discrete units suitable as unitary dosages for human subjects, each unit containing a predetermined quantity of TG2 inhibitor calculated in an amount sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Depending on the patient and condition being treated and on the administration route, the TG2 inhibitor is administered in dosages of 1 mg to 2000 mg/kg body weight per day, e.g. about 100, 500, 1000, 10,000 mg/day for an average person. Durations of the regimen may be from: 1×, 2×3× daily; and in a combination regimen may be from about 1, about 7, about 14, etc. days prior to administration of second agent. Dosages are appropriately adjusted for pediatric formulation. Those of skill will readily appreciate that dose levels can vary as a function of the specific inhibitor, the diet of the patient and the gluten content of the diet, the severity of the symptoms, and the susceptibility of the subject to side effects. Some of the inhibitors of the invention are more potent than others. Preferred dosages for a given inhibitor are readily determinable by those of skill in the art by a variety of means. A preferred means is to measure the physiological potency of a given compound.

Various methods for administration are employed in the practice of the invention. In one preferred embodiment, oral administration, for example with meals, is employed. The dosage of the therapeutic formulation can vary widely, depending upon the nature of the disease, the frequency of administration, the manner of administration, the clearance of the agent from the patient, and the like. The initial dose can be larger, followed by smaller maintenance doses. The dose can be administered as infrequently as weekly or biweekly, or more often fractionated into smaller doses and administered daily, with meals, semi-weekly, and the like, to maintain an effective dosage level.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Example 1

Purification of recombinant human TG2. TG2 was purified from eight 1 L cultures of the BL21 strain of E. coli expressing a plasmid encoding recombinant, 6×-His tagged human tissue transglutaminase 2 (TG2). The enzyme was isolated from cell lysates using nickel affinity chromatography. Following a buffer exchange, TG2 was purified further by ion-exchange (HiTrap-Q) chromatography using an Akta-FPLC system (Amersham Biosciences). Fractions containing purified TG2 were pooled, exchanged into low salt buffer (20 mM Tris-Cl, 1 mM EDTA, 1 mM DTT, pH 7.2), supplemented with 10% glycerol, flash frozen in 100 μL aliquots and stored at −80° C.

Assays of tissue transglutaminase activity. The transamidation activity of TG2 was measured as described by Slaughter, et al. Anal Biochem, 1992. 205(1): p. 166-71. Briefly, 96-well Maxisorp plates (Nunc, Inc.) were coated with 15 mg/mL N′—N′-dimethylated casein overnight at 4° C. TG2 (20 nM) was incubated in a final volume of 200 μL reaction buffer (100 mM Tris, pH 8.5, 5 mM CaCl₂, 10 mM dithiothreitol, 200 μM 5-(biotinamido)pentylamine (5-BP), 3.3% DMSO) with or without potential inhibitors for 30 minutes. 5-BP incorporated by TG2 was visualized by binding of a streptavidin-alkaline phosphatase conjugate (Sigma) and kinetic measurement of the dephosphorylation of pNPP was monitored at 410 nm using a ThermoMax microplate reader (Molecular Devices). The deamidation activity of TG2 was measured essentially as described previously (Piper, et al. Biochemistry, 2002. 41(1): p. 386-93). Briefly, TG2 was incubated in the presence of DMSO alone or various inhibitors in reaction buffer (200 mM MOPS pH 7.2, 10 mM CaCl₂, 1 mM EDTA, 0.25 U/mL glutamate dehydrogenase (GDH), 10 mM α-ketoglutarate, 300 μM NADH, and various concentrations of the dipeptide analog Z-Gln-Gly). TG2-catalyzed production of ammonium ions was monitored through the coupled oxidation of NADH by GDH. NADH levels were monitored by fluorescence (excitation: 341 nm; emission 451 nm) and were standardized with known amounts of NADH. During enzymatic reactions, NADH levels were monitored kinetically using a Fluoroskan II (Labsystems, Inc.) 96-well plate reader and a lag time of four minutes.

IC₅₀ values for inhibition of TG2 by compounds of Formulas I-IX are presented in Table I below. These values were determined through analysis of the log(concentration)-response relationships. Compound CSRF-21 interfered with the fluorescent NADH-based assay and its IC₅₀ was determined using the 5-(biotinylated)pentylamine assay.

TABLE 1
IC₅₀ values for TG2 inhibitors.

| Compound | IC₅₀ |
|---|---|
| CSRF-2 | 36 μM |
| CSRF-3 | 30 μM |
| CSRF-6 | 17 μM |
| CSRF-7 | 30 μM |
| CSRF-8 | 150 μM |
| CSRF-13 | 250 μM |
| CSRF-14 | 16 μM |
| CSRF-17 | 200 μM |
| CSRF-18 | 23 μM |
| CSRF-20 | 110 μM |
| CSRF-21 | 10 μM |
| CSRF-24 | 450 μM |

Structures of TG2 inhibitors of Formulas I-IX.
CSRF-2

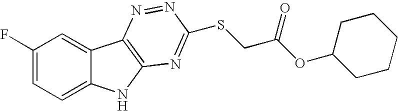

CSRF-3

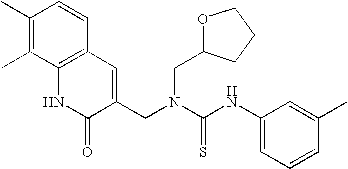

CSRF-6

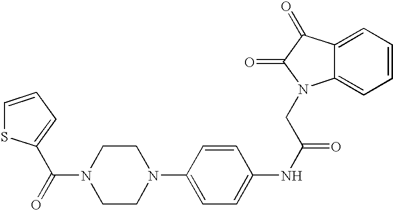

CSRF-7

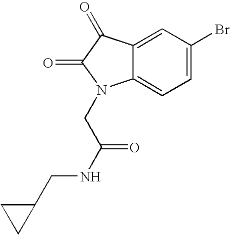

CSRF-8

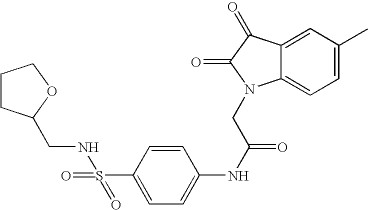

CSRF-13

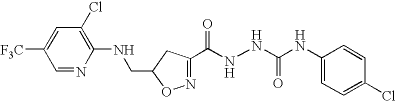

CSRF-14

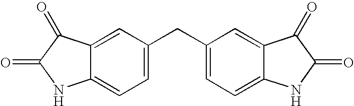

CSRF-17

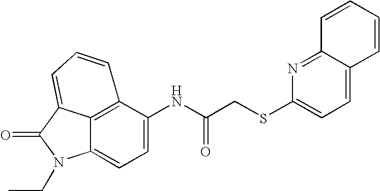

CSRF-18

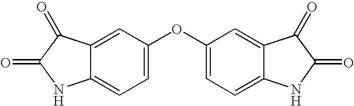

CSRF-20

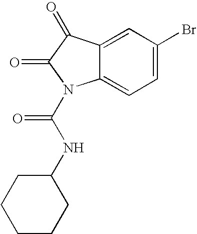

CSRF-21

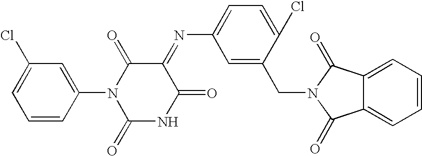

CSRF-24

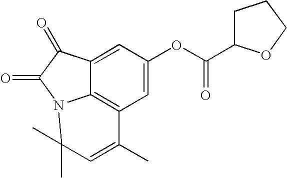

### Example 2

### Use of tTGase Inhibitors for Treating Neurologic Cancers

The compounds set forth in Table 1 are used in the treatment of neurologic cancers in an animal model. The previously described TG2 inhibitor, KCC009 ((S)-[3-(4-hydroxyphenyl)-2-N-(phenylmethyloxycarbonyl)aminopropanoic acid N′-(3′-bromo-4′,5′-dihydro-5′-isoxalyl)methylamide) has been shown to be active in a mouse model for neurologic cancer at concentrations of 12.5-50 mg/kg. Based on FDA guidelines for calculation of human equivalent dose, the HED for KCC009 is 1-4 mg/kg. Inhibitors of the invention are administered in a mouse model at a dose range from 1 to 100 mg/kg, which correspond to a human equivalent dose range of 0.08 to 8 mg/kg.

Therapeutic doses of a compound of the invention are administered to an animal with intracranial glioblastomas. DBT tumors are injected intracranially in mice. As judged by MRI, the mice consistently establish intracranial tumors after 7-10 days following injections, which, if untreated, caused death by 2-3 weeks. In an initial dose-finding experiment, cohorts of mice are administered 4 daily doses of vehicle or inhibitor starting on day 11 after intra-cranial injections. Twenty-four hours later, the mice are sacrificed and tumors analyzed. Gross tumor dissections reveal the extent of tumor growth. Based on these findings, a dose is selected for subsequent evaluation in mice harboring intracranial DBT tumors.

Mice harboring DBT tumors are treated either with 10 mg/kg BCNU alone or BCNU+inhibitor. Four daily drug doses are administered intraperitoneally starting on day 11. Tumors are harvested for immuno-histological analysis 24 h after the last dose; the controls show linear strands of fibronectin in the ECM.

To verify the inhibition of tumor associated TG2, mice are treated with four daily doses of BCNU alone or BCNU+inhibitor, and are sacrificed either 15 min or 24 h after the last dose, and tumor associated TG2 activity is measured.

In addition to evaluating the chemosensitizing potential, the compounds' activity as a radiosensitizer is also evaluated. For this, clonogenic assays are used to measure colony formation after a single radiation treatment. The studies are performed in triplicate with varying doses of XRT and number of cells plated.

To highlight the clinical relevance of TG2 inhibition among tumors of neurological origin, TG2 activity was assessed in tissue samples from astrocytomas (Grade III/IV and IV/IV) and meningiomas (Grades I/IV; II/IV; and III/IV). TG2 activity was measured from 50 μM thick sections of brain tumor specimens collected from the operating room. Each specimen was coded and stored in the Tumor Repository. Samples analyzed included 4 normal brain sample, 6 anaplastic astrocytomas (Grade III/IV); 9 glioblastomas (Grade III/IV), 7 typical meningiomas (Grade I/IV); 5 atypical meningiomas (Grade II/IV); and 3 anaplastic meningiomas (Grade III/IV). The results showed that astrocytomas, glioblastomas and especially meningiomas had elevated TG2 activity and are therefore candidates for inhibitor therapy.

To verify the therapeutic utility of the inhibitor against meningiomas, IOMM-Lee meningioma cells are treated with 20 Gy of radiation with and without TG2 inhibitor. To demonstrate the relationship between TG2 inhibition and extracellular matrix assembly, immunoprecipitated fibronectin from the different cultures are stained with anti-TG2 antibodies via Western blot analysis.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in protein structure without affecting the biological action in kind or amount. All such modifications are intended to be included within the scope of the appended claims.

What is claimed is:

1. A method of treating cancer, the method comprising:
administering to a patient an effective dose of a TG2 inhibitor having the formula selected from Formula I-III and V-IX: 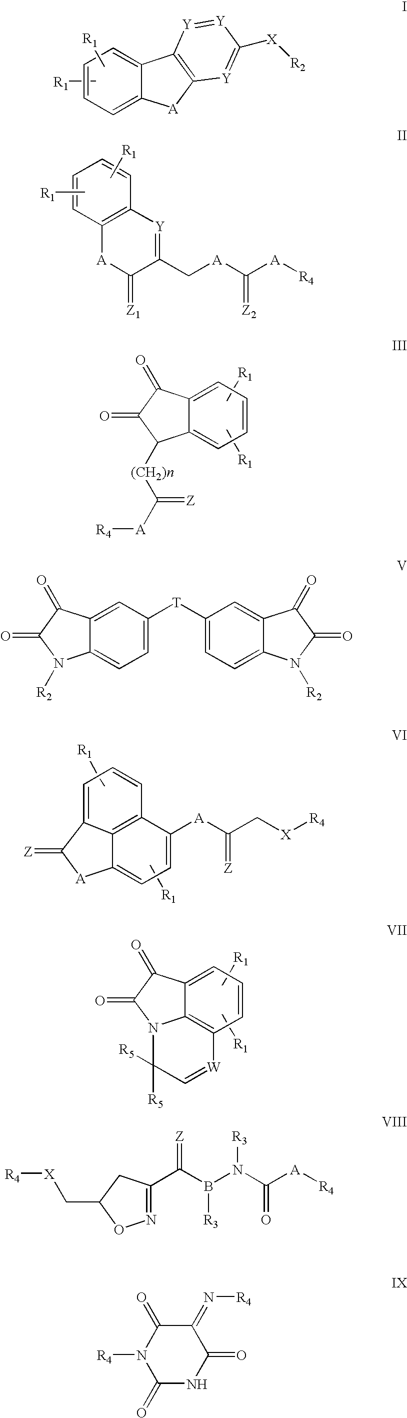
wherein in each Formula, R₁ is independently and in each instance independently hydrogen, halogen, nitro, sulfonyl, acyl, alkoxy, alkyl;
A is independently and in each instance O, S or preferably N—R₃, where R₃ is H, optionally substituted lower alkyl, aryl, heteroaryl, alkaryl;
X is independently and in each instance CH₂, CF₂, CH(OH), CH(O-lower alkyl), C(O), N—R₃, O, S, S(O) or S(O)₂;
Y is independently and in each instance C—R₁, or N. X is CH₂, CH(OH), CH(O-lower alkyl), N—R₃, O, S, S(O) or S(O)₂:
R₂ is independently and in each instance alkyl, aryl, heteroaryl, acetic ester
(CH₂C(O)OR₃) or an acetamide (CH₂C(O)NR₃R₃);
Z is independently and in each instance O or S, and R₄ is hydrogen, optionally substituted aryl, heteroaryl alkyl, or alkaryl;
T is X or a linker;
B is CH or N;
n is 0 or 1;
W is N or C—R₃;
R₅ is independently and in each instance H, optionally substituted lower alkyl, aryl, heteroaryl, alkaryl, a carboxylic ester (C(O)OR₃) or a carboxamide (C(O)NR₃R₃).

2. The method of claim 1, wherein said cancer is a solid tumor.

3. The method according to claim 2, wherein said solid tumor is a neural tumor.

4. The method according to claim 1, wherein said TG2 inhibitor is administered in combination with a cytotoxic agent.

5. The method according to claim 4, wherein said cytotoxic agent is selected from the group consisting of alkylating agents, antimitotics, anthracyclines, microtubule stabilizing agents, and metal complexes.

6. The method according to claim 1, wherein said TG2 inhibitor is administered in combination with radiation.

7. The method according to claim 1, wherein said TG2 inhibitor is selected from the group consisting of:

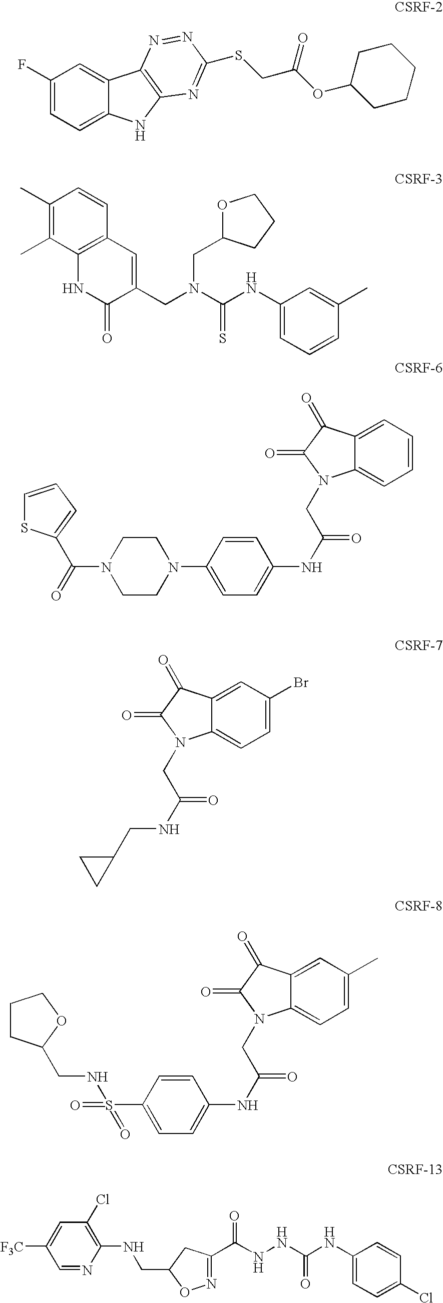

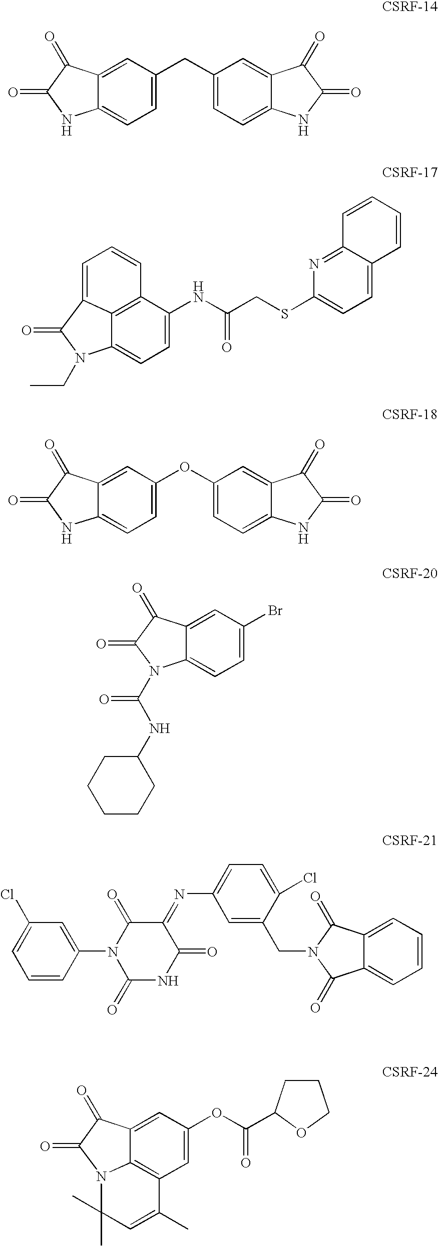

8. A method of treating Celiac Sprue and/or dermatitis herpetiformis, the method comprising:
administering to a patient an effective dose of a TG2 inhibitor having a formula selected from Formula I-III and V-IX: 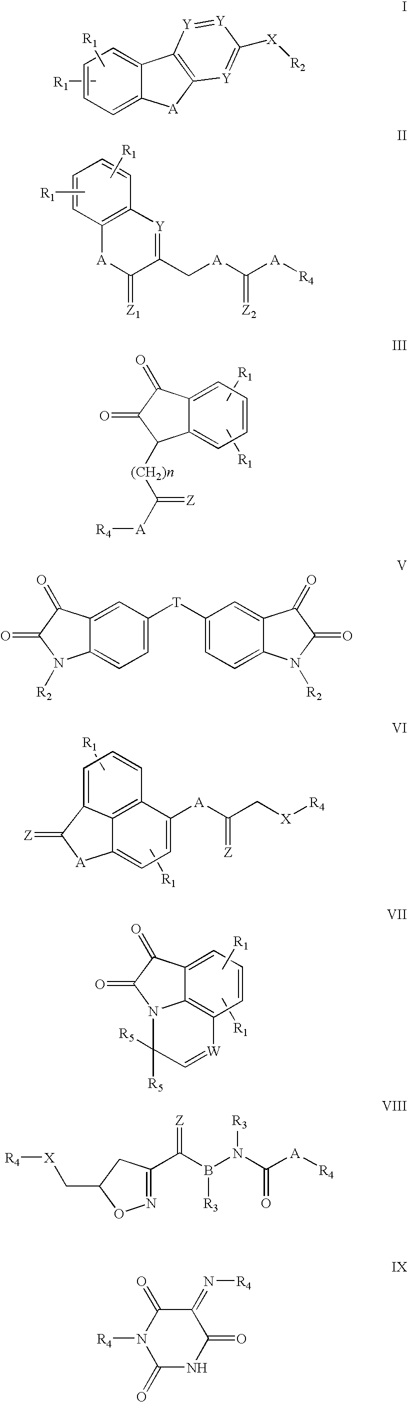
wherein in each Formula, R₁ is independently and in each instance independently hydrogen, halogen, nitro, sulfonyl, acyl, alkoxy, alkyl;
A is independently and in each instance O, S or preferably N—R₃, where R₃ is H, optionally substituted lower alkyl, aryl, heteroaryl, alkaryl;
X is independently and in each instance CH₂, CF₂, CH(OH), CH(O-lower alkyl), C(O), N—R₃, O, S, S(O) or S(O)₂;
Y is independently and in each instance C—R₁ or N. X is CH₂, CH(OH), CH(O-lower alkyl), N—R₃, O, S, S(O) or S(O)₂:
R₂ is independently and in each instance alkyl, aryl, heteroaryl, acetic ester
(CH₂C(O)OR₃) or an acetamide (CH₂C(O)NR₃R₃);
Z is independently and in each instance O or S, and R₄ is hydrogen, optionally substituted aryl, heteroaryl alkyl, or alkaryl;
T is X or a linker;
B is CH or N;
n is 0 or 1;
W is N or C—R₃;
R₅ is independently and in each instance H, optionally substituted lower alkyl, aryl, heteroaryl, alkaryl, a carboxylic ester (C(O)OR₃) or a carboxamide (C(O)NR₃R₃).

9. The method of claim 8, wherein said TG2 inhibitor is administered with a glutenase.

10. The method according to claim 8, wherein said TG2 inhibitor is administered orally.

11. The method according to claim 8, wherein said TG2 inhibitor is contained in a formulation that comprises an enteric coating.

12. The method according to claim 9, wherein said TG2 inhibitor is selected from the group consisting of:

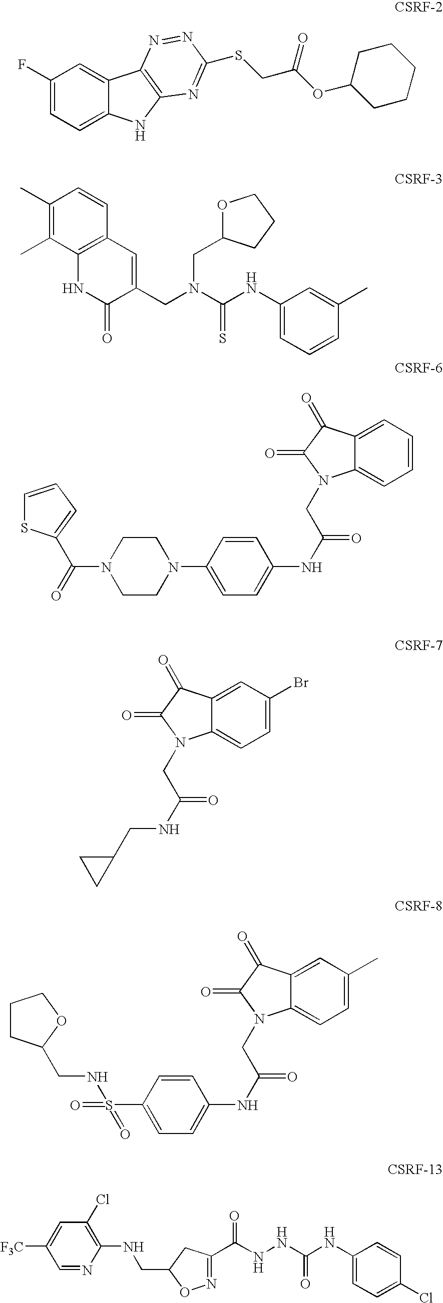

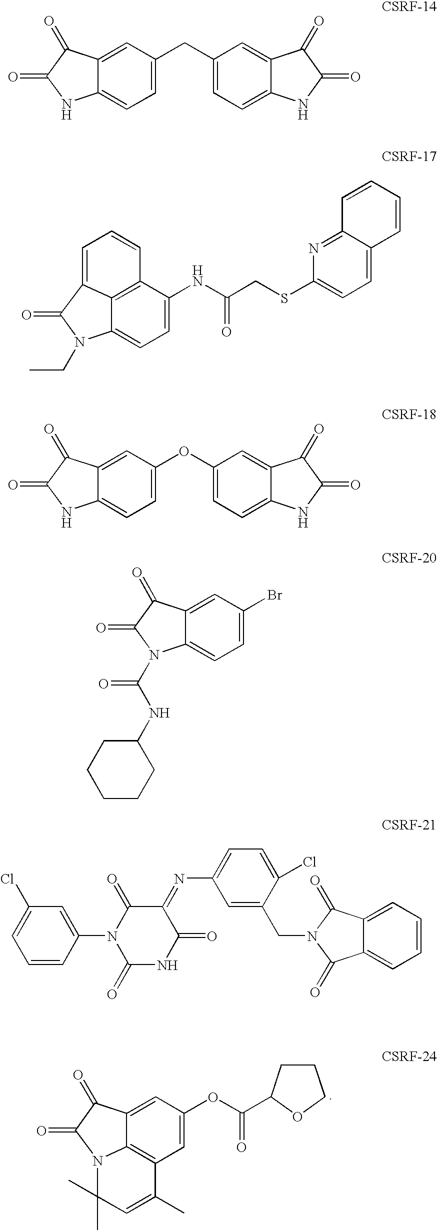

13. A pharmaceutical formulation comprising:
an effective dose of a TG2 inhibitor, wherein said TG2 inhibitory moiety has a formula selected from Formula I-III and V-IX: 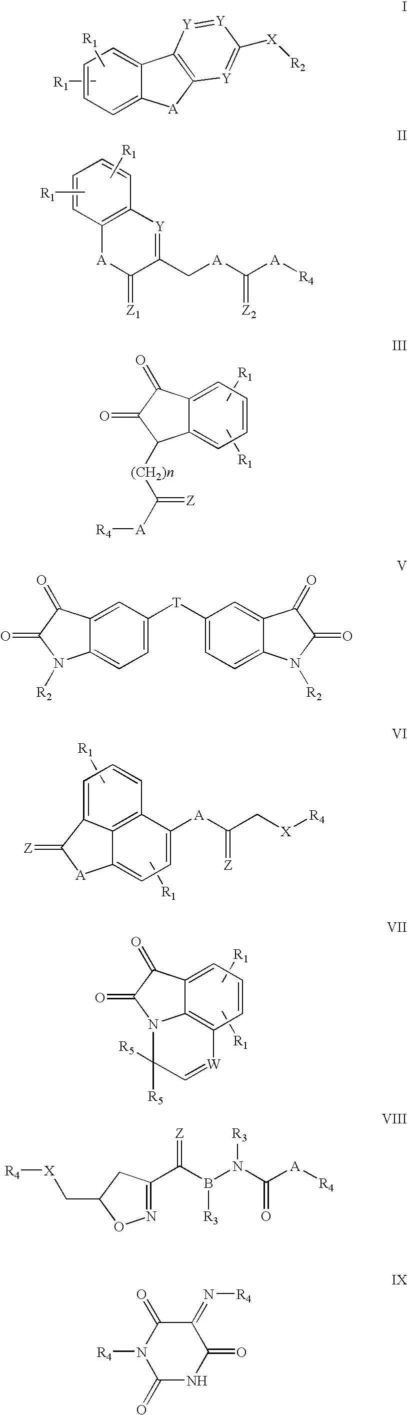
wherein in each Formula, R₁ is independently and in each instance independently hydrogen, halogen, nitro, sulfonyl, acyl, alkoxy, alkyl;
A is independently and in each instance O, S or preferably N—R₃, where R₃ is H, optionally substituted lower alkyl, aryl, heteroaryl, alkaryl;
X is independently and in each instance CH₂, CF₂, CH(OH), CH(O-lower alkyl), C(O), N—R₃, O, S, S(O) or S(O)₂;
Y is independently and in each instance C—R₁, or N. X is CH₂, CH(OH), CH(O-lower alkyl), N—R₃, O, S, S(O) or S(O)₂:
R₂ is independently and in each instance alkyl, aryl, heteroaryl, acetic ester (CH₂C(O)OR₃) or an acetamide (CH₂C(O)NR₃R₃);
Z is independently and in each instance O or S, and R₄ is hydrogen, optionally substituted aryl, heteroaryl alkyl, or alkaryl;
T is X or a linker;
B is CH or N;
n is 0 or 1;
W is N or C—R₃;
R₅ is independently and in each instance H. optionally substituted lower alkyl, aryl, heteroaryl, alkaryl, a carboxylic ester (C(O)OR₃) or a carboxamide (C(O)NR₃R₃);
and a pharmaceutically acceptable excipient.

14. The formulation of claim 13, wherein said TG2 inhibitor is selected from the group consisting of: 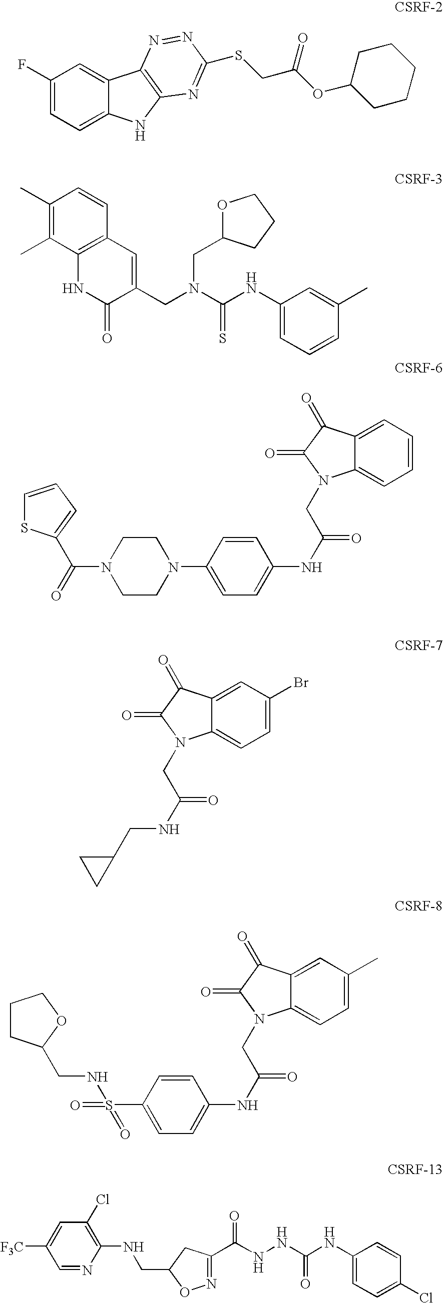 [chemical structure]
